(19) United States
(12) Patent Application Publication
(10) Pub. No.: US 2012/0184750 A1
(43) Pub. Date: Jul. 19, 2012

(54) PROCESS FOR THE PREPARATION OF OLMESARTAN MEDOXOMIL

(75) Inventors: Ashwini Kumar Kapoor, Aurangabad (IN); Hiten Sharadchandra Mehta, Ahmedabad (IN); Asok Nath, Gurgaon (IN); Mohan Prasad, Gurgaon (IN)

(73) Assignee: RANBAXY LABORATORIES LIMITED, New Delhi, Delhi (IN)

(21) Appl. No.: 13/321,231

(22) PCT Filed: May 20, 2010
(86) PCT No.: PCT/IB10/52260

§ 371 Date: Apr. 10, 2012

(30) Foreign Application Priority Data
May 20, 2009 (IN) 1037/DEL/2009

(51) Int. Cl. C07D 405/14

(52) U.S. Cl. 548/253

(57) ABSTRACT

The present invention provides an improved process for the preparation of olmesartan medoxomil, which is free of OLM-acid and has lower amount of eliminate and acetic acid impurity.

### FIELD OF THE INVENTION

The present invention provides an improved process for the preparation of olmesartan medoxomil, which is free of OLM-acid and has lower amount of eliminate and acetic acid impurity.

### BACKGROUND OF THE INVENTION

Antihypertensive agents belong to a group of angiotensin II antagonists which are generally referred to as “sartans”. These include olmesartan, candesratan, irbesartan, losartan and valsartan. They act as powerful vasodilators and work by blocking the action of angiotensin II receptor. U.S. Pat. No. 5,616,599 (the '599 patent) covers olmesartan medoxomil, 2,3-dihydroxy-2-butenyl-4-(1-hydroxy-1-methylethyl)-2-propyl-1-[p-(o-1H-tetrazol-5-ylphenyl)benzyl]imidazole-5-carboxylate, cyclic 2,3-carbonate, having the structural Formula 1:

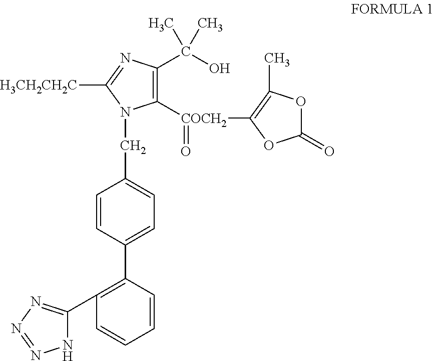

Olmesartan medoxomil (Benicar®) is a prodrug that is hydrolyzed during absorption and is a selective AT1 subtype angiotensin II receptor antagonist.

Several methods of preparing olmesartan medoxomil are known such as those described in U.S. Pat. Nos. 5,616,599 and 5,763,619; U.S. Publication Nos. 2005/0119488; 2006/0148870; 2006/0069141; 2006/0074117; 2007/0054948; 2010/0076200; and PCT Publication Nos. WO 2006/050922, WO 2007/048361 and WO 2005/021535, which are incorporated herein by reference.

The '599 patent describes a process for preparing olmesartan medoxomil comprising deprotecting trityl olmesartan medoxomil (MTT) with 70% aqueous acetic acid at 60° C. The '599 patent process produces a gel-like product, which is difficult to handle in an industrial process and achieves a lower yield of olmesartan medoxomil containing 2.2% OLM-acid per area percent HPLC. Benicar® contains 0.3% OLM-acid per area percent HPLC.

U.S. Publication No. 2006/0069141 describes a process for the preparation of olmesartan medoxomil comprising contacting trityl olmesartan medoxomil with an acid, such as sulfuric acid, water and water miscible organic solvent such as acetone. The process of the '141 application yields olmesartan medoxomil containing about 0.89% OLM-acid.

U.S. Publication Nos. 2006/0074117 and 2010/0076200 describe a process for the purifying olmesartan medoxomil comprising mixing a solution of olmesartan medoxomil in a C₃₋₆ ketone followed by addition of water. The process of the 2006/0074117 and 2010/0076200 applications yield olmesartan medoxomil with less than 0.03% OLM acid. U.S. Publication No. 2007/0054948 covers olmesartan medoxomil with less than about 0.12% area by HPLC OLM-acid.

The methods described in aforementioned references may involve large amount of solvents for the final purification, followed by chromatography, multiple extractions or azeotropic distillation. Moreover, the process described therein may involve the use of strong corrosive acids or refluxing conditions, which are difficult to handle in an industrial scale process.

Therefore, there is a need for an improved process which is simple, cost effective and produces pure olmesartan medoxomil in better yields with a lower amount of impurity.

### SUMMARY OF THE INVENTION

In one general aspect, the present invention provides for a process for the preparation of olmesartan medoxomil. The process includes: a) mixing a catalytic amount of a strong acid with a solution or suspension of trityl olmesartan medoxomil in a mixture of weak acid and water; b) isolating olmesartan medoxomil; c) dissolving the olmesartan medoxomil obtained from step (b) in a polar organic solvent; and d) isolating pure crystalline olmesartan medoxomil.

Embodiments of the present invention may include one or more of the following features. For example, the strong acid may be perchloric acid, chloric acid, chlorous acid, hypochlorous acid, sulfuric acid, sulfurous acid, nitric acid, phosphoric acid, carbonic acid, hydrochloric acid or trifluoroacetic acid. The catalytic amount of the strong acid may be from about 1 to about 1.5 molar equivalents of trityl olmesartan medoxomil.

The weak acid may be acetic acid. The acetic acid may include water in the ratio of about 1:1.

The process may further include raising the temperature of reaction mixture in the step a) to about 25° C. to about 35° C. The process may also include heating the reaction mixture in step c) at about 40° C. to a reflux temperature of the solvent.

The polar organic solvent may be nitriles, ketones or alcohols. The polar organic solvent may be acetonitrile, acetone, ethylmethylketone, 2-pentanone, 3-pentanone, ethanol or methanol.

In another general aspect there is provided a process for the purification of olmesartan medoxomil. The process includes: a) dissolving olmesartan medoxomil free of OLM-acid impurity in polar organic solvent; and b) isolating pure crystalline olmesartan medoxomil.

Embodiments of the present invention may include one or more of the following features. For example, the process may further include heating the reaction mixture in step a) at about 40° C. to a reflux temperature of the solvent.

The polar organic solvent may be nitriles, ketones or alcohols. The polar organic solvent may also be acetonitrile, acetone, ethylmethylketone, 2-pentanone, 3-pentanone, ethanol or methanol.

In another general aspect the present invention provides for olmesartan medoxomil free of acetic acid and/or OLM-acid.

In yet another general aspect the present invention provides for olmesartan medoxomil containing less than about 0.05% OLM-eliminate impurity.

In a final general aspect, the present invention provides for olmesartan medoxomil having no detectable amount of impurities at RRT 0.34 and 1.15 when measured by HPLC area percentage.

### DETAILED DESCRIPTION OF THE INVENTION

The present invention provides an improved process for the preparation of olmesartan medoxomil comprising the steps of:
a) adding a solution or suspension of trityl olmesartan medoxomil to a mixture of weak acid and water;
b) adding a strong acid in catalytic amounts; or adding trityl olmesartan medoxomil to a solution or suspension of weak acid, water and strong acid in catalytic amounts;
c) isolating olmesartan medoxomil;
d) dissolving the olmesartan medoxomil obtained from step (c) in a polar organic solvent; and
e) isolating pure crystalline olmesartan medoxomil.

Trityl olmesartan medoxomil can be prepared by following any methods known to a person of ordinary skill in the art including the references disclosed in the background section of this invention.

Trityl olmesartan medoxomil may be added to a mixture of a weak acid and water or a mixture of two or more acids and water.

The weak acid used for preparing a solution or suspension of trityl olmesartan medoxomil with water may be an organic acid, preferably acetic acid. The ratio of water to the organic acid e.g., acetic acid, is preferably about 2:1 to about 1:2, and more preferably about 1:1. A catalytic amount of a strong acid may be added to the solution or suspension. The pH of a strong acid may range from 0 to 4.

Suitable strong acids include perchloric acid, chloric acid, chlorous acid, hypochlorous acid, sulfuric acid, sulfurous acid, nitric acid, nitrous acid, phosphoric acid, carbonic acid, hydrochloric acid or trifluoroacetic acid. Sulfuric acid is preferred. Preferably the catalytic amount of acid used is about 1 to about 2 molar equivalents, more preferably about 1 to 1.5 molar equivalents and most preferably about 1 mole equivalent of the trityl olmesartan medoxomil.

The addition of a strong acid may require a time period of from 10 to 25 minutes. The temperature of the reaction mixture may be cooled to about 5° C.-15° C. The reaction mixture containing trityl olmesartan medoxomil may be stirred for about 25 minutes to 4 hours. The detritylation reaction may be carried out at a temperature range of about 0° C. to about 35° C., preferably at room temperature.

In a preferred embodiment, the acid or acid mixture removes triphenylcarbinol by forming precipitates without the formation of any acid salt of olmesartan medoxomil. The acetone may be added prior to the separation of triphenyl carbinol to avoid the formation of undesirable impurities. Preferably the amount of acetone used is about ¼ volume of the acid-water mixture. Precipitation of the triphenylcarbinol involves the formation of distinct particles of the precipitates suspended in the suspension or collected at the bottom of the vessel containing the solution.

The precipitates of the triphenylcarbinol can be removed from the solution by any means known in the prior-art, such as filtration or centrifugation.

After separating the triphenylcarbinol, the olmesartan medoxomil solution is contacted with a base. The base is used here to neutralize the catalytic amount of the acid used. Suitable bases include alkali and alkaline earth metal hydroxides, carbonates and hydrogen carbonates. Particularly used bases include sodium hydroxide, potassium hydroxide, calcium hydroxide, magnesium hydroxide, sodium carbonate, potassium carbonate, calcium carbonate, sodium bicarbonate and potassium bicarbonate. Potassium carbonate and specifically sodium carbonate are preferred.

The isolation of the crude olmesartan medoxomil free of OLM-acid involves the extraction of the reaction mixture after contacting with the base in halogenated solvent.

Suitable examples of the halogenated solvents include chloroform, dichloromethane, dichloroethane and the like. Preferably, dichloromethane is used for extraction. Solvent is recovered by the methods known in the art including, for example rotatory evaporation under vacuum or distillation.

The product obtained after the solvent recovery is in the form of an oil. The oily product is dissolved in water miscible solvents, including dioxane, tetrahydrofuran, ketones, alcohols or acetonitrile. Preferably, acetonitrile is used. The dissolution step is repeated again with the product obtained after the first dissolution in a water miscible solvent to obtain crystallized olmesartan medoxomil free of OLM-acid and having low levels of impurity.

According to another aspect, the present invention provides a process for purifying olmesartan medoxomil. The process includes the steps of:
a) preparing a solution of olmesartan medoxomil free of OLM-acid in a polar organic solvent; and
b) isolating pure crystalline olmesartan medoxomil.

Suitable polar organic solvents include nitriles, ketones and alcohols. Preferred solvents are acetonitrile, acetone, ethylmethylketone, 2-pentanone, 3-pentanone, ethanol and methanol. Preferably the polar organic solvent used is a ketonic solvent such as acetone. A preferable amount of ketone is at least about 4 volumes ketone to about 1 gram of solid olmesartan medoxomil, more preferably at least about 3 volumes ketone to about 1 gram of solid olmesartan medoxomil and the most preferably at least about 2 volumes ketone to about 1 gram of solid olmesartan medoxomil.

The process may further include the step of heating the dissolution of crude olmesartan medoxomil in polar organic solvent. The solution of olmesartan medoxomil in polar organic solvent is preferably heated to about 40° C. to reflux temperature, more preferably from about 50° C. to about reflux temperature.

The solution so obtained may be cooled to about 25° C.-35° C. Charcoal is added to the solution over a time period of about 20 minutes to 35 minutes. Charcolized solution is filtered through hyflobed followed by washing with polar organic solvent. The amount of polar organic solvent used for washing is preferably about 0.2 volume to about 0.4 volume of the polar organic solvent, more preferably 0.2 volume. The process further includes the step of condensation of the combined filtrate to about 1 volume of the total volume at 35° C.-45° C. The condensed solution may be cooled from about 15° C. to about 25° C. and stirred for about 3-4 hours.

The pure crystalline olmesartan medoxomil free of OLM-acid and having low levels of eliminate and acetic acid impurity can be recovered by any means known to a person of ordinary skill in the art, including for example, centrifugation or filtration which may further include washing with polar organic solvent. The crystalline olmesartan medoxomil can be dried at about 45° C. to 55° C. by any drying methods such as vacuum or air drying.

According to a preferred embodiment, olmesartan medoxomil obtained by the processes of the present invention has no detectable amount of acetic acid and/or OLM-acid impurities.

One embodiment of the present invention provides a substantially pure olmesartan medoxomil, wherein the term substantially pure refers to olmesartan medoxomil free of OLM-acid, having lower amount of eliminate and acetic impurity in the final product.

Another embodiment of the present invention provides substantially pure olmesartan medoxomil containing less than about 0.1% of the eliminate impurity, more preferably less than about 0.07%, and the most preferably less than about 0.05%.

Yet another embodiment of the present invention provides substantially pure olmesartan medoxomil having lower amount of acetic acid as the potential impurity.

According to another embodiment, olmesartan medoxomil obtained according to the present invention has a HPLC purity of greater than 99%, more preferably greater than about 99.77%.

In a particular embodiment, olmesartan medoxomil does not have detectable level of impurities at RRT 0.34 and 1.15 when measured by HPLC area percentage.

Olmesartan medoxomil so obtained may be used for preparing a pharmaceutical composition with a pharmaceutically acceptable excipient, which can be used for the treatment of hypertension in human.

In the following section embodiments are described by way of examples to illustrate the process of invention. However, these are not intended in any to limit the scope of present invention. Several variants of these examples would be evident to persons ordinarily skilled in the art.

### EXAMPLES

### Example 1

### Preparation of Olmesartan Medoxomil

Trityl olmesartan medoxomil (100 gm) was added to a mixture of acetic acid, water (1:1; 400 mL) and the suspension was brought to temperature of 10° C.-15° C. Sulfuric acid (12.2 gm) (1 mol equivalent) was charged to the reaction mixture slowly at 10° C.-15° C. in 15 minutes. The temperature of the reaction mixture was raised to 25° C.-30° C., stirred for 45 minutes and filtered to remove triphenyl carbinol. Sodium carbonate solution (25% w/v, 100 mL) was charged to the filtrate and the product was extracted with dichloromethane (500 mL) followed by recovery of the solvent. The product was isolated, recrystallized using acetonitrile (300 mL), filtered, washed and dried under reduced pressure to obtain crude olmesartan medoxomil.

### Yield: 80%

HPLC purity: 99.77%

### OLM-acid: Not Detectable

### OLM-Eliminate: 0.05%

Acetic acid content: Not Detectable

### Example 2

### Preparation of Olmesartan Medoxomil

A mixture of trityl olmesartan medoxomil in acetic acid and water (1:1, 400 mL) and sulfuric acid (12.2 gm) (1 mol equivalent) was stirred at 25° C.-30° C. for 45-60 minutes. Triphenylcarbinol was filtered and the filtrate was washed with acetic acid and water mixture (1:1, 50 mL). Sodium carbonate solution (25% w/v, 100 mL) was charged to the filtrate and the product was extracted with dichloromethane (500 mL) followed by recovery of the solvent. The product was isolated, recrystallized using acetonitrile (300 mL), filtered, washed and dried under reduced pressure to obtain crude olmesartan medoxomil.

### Yield: 90%

HPLC purity: 99.29%

### OLM-acid: Not Detectable

### OLM-Eliminate: 0.07%

Acetic acid content: Not Detectable

### Example 3

### Purification of Olmesartan Medoxomil (Crude)

Crude olmesartan medoxomil (10 gm) was dissolved in acetone (2000 mL) at 55° C.-60° C. and solution was cooled to 45° C. The solution was charcolized at the same temperature for 30 minutes. The charcolized reaction mixture was filtered at 40° C. through hyflobed and washed with acetone (2×100 mL). The combined filtrate was concentrated to 1000 mL of the total volume at 40° C.-45° C., cooled to 25° C. and stirred at 25° C.-30° C. for 2 hours. The product was collected after filtration, washed with acetone (2×50 mL) and dried under reduced pressure at 45° C.-50° C.

### Yield: 90%

### Example 4

### Impurity Profile Determination of Olmesartan Medoxomil

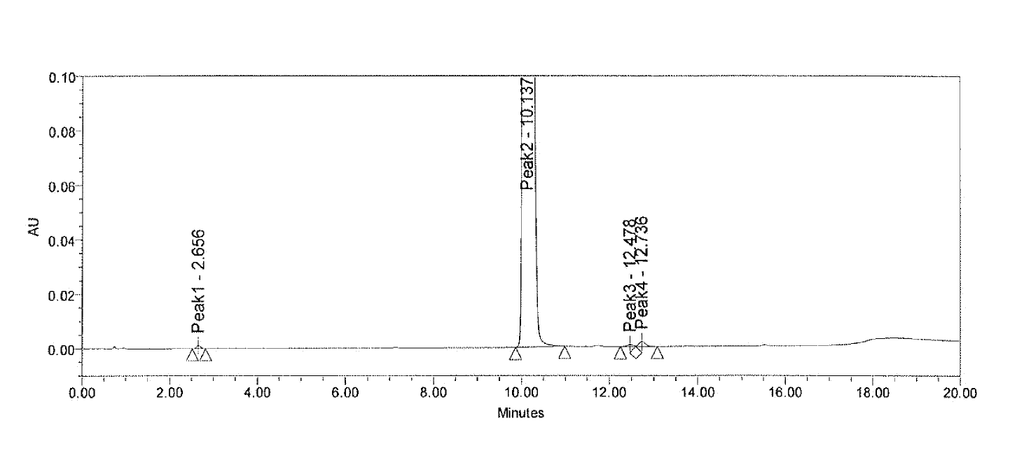

As per the analytical method used for the validation and quantification of the impurities in olmesartan medoxomil of the present invention, hydrolyzed impurity i.e., OLM-acid and has been removed completely and other potential impurity, such as eliminate and methylpropyl analog impurity have been reduced to low levels when analyzed by HPLC assay with respect to their respective RRT values i.e., 0.34 for OLM-acid, 1.23 for eliminate impurity and 1.15 for Methylpropyl analog impurity. In a particular embodiment, olmesartan medoxomil does not have detectable levels of impurities when measured by HPLC at RRT 0.34 and 1.15 (FIG. 1).

1. A process for the preparation of olmesartan medoxomil comprising:
a) mixing catalytic amounts of a strong acid with a solution or suspension of trityl olmesartan medoxomil in a mixture of weak acid and water;
b) isolating olmesartan medoxomil;
a) dissolving the olmesartan medoxomil obtained from step (b) in a polar organic solvent; and
b) isolating pure crystalline olmesartan medoxomil.

2. The process according to claim 1, wherein the strong acid is selected from the group consisting of perchloric acid, chloric acid, chlorous acid, hypochlorous acid, sulfuric acid, sulfurous acid, nitric acid, phosphoric acid, carbonic acid, hydrochloric acid and trifluoroacetic acid.

3. The process according to claim 1, wherein about 1 to about 1.5 molar equivalents of strong acid with respect to the quantity of trityl olmesartan medoxomil is used.

4. The process according to claim 1, wherein the weak acid is acetic acid.

5. The process according to claim 4, wherein the mixture of acetic acid and water is in the ratio of about 1:1 by volume.

6. The process according to claim 1, further comprising raising the temperature of reaction mixture in the step a) to about 25° C. to about 35° C.

7. The process according to claim 1, further comprising heating the reaction mixture in step c) at about 40° C. to a reflux temperature of the solvent.

8. The process according to claim 1, wherein the polar organic solvent comprises nitriles, ketones or alcohols.

9. The process according to claim 1, wherein the polar organic solvent comprises acetonitrile, acetone, ethylmethylketone, 2-pentanone, 3-pentanone, ethanol or methanol.

10. A process for the purification of olmesartan medoxomil comprising:
a) dissolving olmesartan medoxomil free of OLM-acid impurity in polar organic solvent; and
b) isolating pure crystalline olmesartan medoxomil.

11. The process according to claim 10, further comprising heating the reaction mixture in step a) at about 40° C. to a reflux temperature of the solvent.

12. The process according to claim 10, wherein the polar organic solvent comprises nitriles, ketones or alcohols.

13. The process according to claim 10, wherein the polar organic solvent comprises acetonitrile, acetone, ethylmethylketone, 2-pentanone, 3-pentanone, ethanol or methanol.

14. Olmesartan medoxomil free of acetic acid or OLM-acid impurities.

15. Olmesartan medoxomil containing less than about 0.05% OLM-eliminate impurity.

16. Olmesartan medoxomil having no detectable amount of impurities at RRT 0.34 and 1.15 when measured by HPLC area percentage.
